(19) United States
(12) Patent Application Publication
(10) Pub. No.: US 2025/0152029 A1
(43) Pub. Date: May 15, 2025

(54) PROMOTING GENERALIZATION IN CROSS-DATASET REMOTE PHOTOPLETHYSMOGRAPHY

(71) Applicants: Securiport LLC, Reston, VA (US); University of Notre Dame du Lac, South Bend, IN (US)

(72) Inventors: Nathan Vance, South Bend, IN (US); Jeremy Speth, South Bend, IN (US); Benjamin Sporrer, South Bend, IN (US); Patrick Flynn, South Bend, IN (US); Nathan Carpenter, Reston, VA (US); Juan Manuel SEGURA, Reston, VA (US)

(21) Appl. No.: 18/744,824

(22) Filed: Jun. 17, 2024

Related U.S. Application Data
(60) Provisional application No. 63/521,433, filed on Jun. 16, 2023.

(51) Int. Cl. A61B 5/024; A61B 5/00; G06T 7/00

(52) U.S. Cl. CPC A61B 5/02433; A61B 5/7485; G06T 7/0012; G06T 2207/10016; G06T 2207/10048; G06T 2207/30004; G06T 2207/30201

(57) ABSTRACT

Systems, devices, methods, and non-transitory computer-readable instructions for determining a physiological signal from a video stream, comprising capturing the video stream of a subject, the video stream including a sequence of frames, processing each frame of the video stream to identify a facial portion of the subject in each frame, determining a periodic physiological signal of the subject from the video stream using a plurality of datasets in which one or more augmentations were applied to the plurality of datasets.

### PRIORITY INFORMATION

This application claims the benefit of the U.S. Provisional Patent Application No. 63/521,433 filed on Jun. 16, 2023, which is hereby incorporated by reference in its entirety.

### FIELD OF THE INVENTION

The embodiments of the present invention generally relate to use of biometrics, and more particularly, to determination of physiological signals from video.

### DISCUSSION OF THE RELATED ART

In general, biometrics may be used to track vital signs that provide indicators about a subject's physical state that may be used in a variety of ways. As an example, for border security or health monitoring, vital signs may be used to screen for health risks (e.g., temperature) or detect deception (e.g., change in pulse or pupil diameter). While sensing temperature is a well-developed technology, collecting other useful and accurate vital signs such as pulse rate (i.e., heart rate or heart beats per minute) or pulse waveform has required physical devices to be attached to the subject. The desire to perform biometric measurement without physical contact has produced some video-based techniques.

Performing reliable pulse rate or pulse waveform estimation from a camera sensor is more difficult than contact plethysmography for several reasons. The change in reflected light from the skin's surface, because of light absorption of blood, is very minor compared to those caused by changes in illumination. Even in settings with ambient lighting, the subject's movements drastically change the reflected light and overpower the pulse signal.

Camera-based vitals estimation is a growing field enabling non-contact health monitoring in a variety of settings. While the number of successful approaches has increased, the size of benchmark video datasets with simultaneous vitals recordings has remained relatively stagnant. It is well-known across the machine learning community that increasing the quantity and diversity of training data is an effective strategy for improving performance.

Collecting remote physiological data is challenging for several reasons. First, recording many hours of high-quality videos results in an unwieldy volume of data. Second, recording a diverse population of subjects with associated medical data is difficult due to privacy concerns. Furthermore, synchronizing contact measurements with video recordings in diverse settings is highly dependent on the researcher's hardware infrastructure and lab setting. Even contact measurements used for ground truth contain noise, making data curation difficult. These difficulties contributing to data scarcity stifle model scaling and robustness.

Accordingly, the inventors have developed systems, devices, methods, and non-transitory computer-readable instructions that enable generalization in cross-dataset remote photoplethysmography.

### SUMMARY OF THE INVENTION

Accordingly, the present invention is directed to generalization in cross-dataset remote photoplethysmography that substantially obviates one or more problems due to limitations and disadvantages of the related art.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the generalization in cross-dataset remote photoplethysmography includes systems, devices, methods, and non-transitory computer-readable instructions for determining a physiological signal from a video stream, comprising capturing the video stream of a subject, the video stream including a sequence of frames, processing each frame of the video stream to identify a facial portion of the subject in each frame, determining a periodic physiological signal of the subject from the video stream using a plurality of datasets in which one or more augmentations were applied to the plurality of datasets.

In the various embodiments, the plurality of datasets includes ground truth data and previously captured video data. In the various embodiments, the ground truth data is captured using pulse oximeter. In the various embodiments, the one or more augmentations is applied to the ground truth data and the previously captured video data. In the various embodiments, the one or more augmentation include at least one of horizontal flip, illumination, and Gaussian noise. In the various embodiments, the periodic physiological signal is heart rate. In the various embodiments, the facial portion is cropped to 64×64 pixels. In the various embodiments, the video stream includes one or more of a visible-light video stream, a near-infrared video stream, a longwave-infrared video stream, a thermal video stream, and an audio stream of the subject. In the various embodiments, each frame of the media stream is cropped to encapsulate a region of interest that includes one or more of a face, cheek, forehead, or an eye. In the various embodiments, combining at least two of a visible-light video stream, a near-infrared video stream, and a thermal video stream are combined into a fused video stream.

It is to be understood that both the foregoing general description and the following detailed description are examples and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates a system for pulse waveform estimation.

FIG. 2 illustrates an overview of the augmentations targeting the temporal domain.

FIG. 3 illustrates an overview of the temporal augmentation framework.

FIGS. 4(A)-(I) illustrate training and validation losses when training RPNet on the three datasets and applying augmentation settings.

FIG. 5 illustrates that speed augmentations reduce learned bias as reflected by a reduced mean error in cross dataset analysis between datasets with differing heart rate bands.

FIG. 6 illustrates that speed augmentations improve the accuracy of the model, reflected by an improved mean absolute error.

### DETAILED DESCRIPTION OF THE EXEMPLARY EMBODIMENTS

Reference will now be made in detail to the embodiments of the present invention, examples of which are illustrated in the accompanying drawings.

Embodiments of user interfaces and associated methods for using a device are described. It should be understood, however, that the user interfaces and associated methods can be applied to numerous devices types, such as a portable communication device such as a tablet or mobile phone. The portable communication device can support a variety of applications, such as wired or wireless communications. The various applications that can be stored (in a non-transitory memory) and executed (by a processor) on the device can use at least one common physical user-interface device, such as a touchscreen. One or more functions of the touchscreen as well as corresponding information displayed on the device can be adjusted and/or varied from one application to another and/or within a respective application. In this way, a common physical architecture of the device can support a variety of applications with user interfaces that are intuitive and transparent.

The embodiments of the present invention provide systems, devices, methods, and non-transitory computer-readable instructions to measure one or more biometrics, including heart-rate and pulse waveform without physical contact with the subject. The embodiments can be used in combination with other biometrics that can include respiration, eye gaze, blinking, pupillometry, face temperature, oxygen level, blood pressure, audio, voice tone and/or frequency, micro-expressions, etc. In the various embodiments, the systems, devices, methods, and instructions collect, process, and analyze video taken in one or more modalities (e.g., visible light, near infrared, longwave infrared, thermal) to provide non-contrastive unsupervised learning of physiological signals from a video signal or video data (e.g., MP4).

As describe herein, the pulse or pulse waveform for the subject's heartbeat may be used as a biometric input to establish features of the physical state of the subject and how they change over a period of observation (e.g., during questioning or other activity). Remote photoplethysmography (rPPG) is the monitoring of blood volume pulse from a camera at a distance. Using rPPG, blood volume pulse from video at a distance from the skin's surface may be detected. The disclosure of U.S. application Ser. No. 17/591,929, entitled “VIDEO BASED DETECTION OF PULSE WAVEFORM”, filed 3 Feb. 2022, is hereby incorporated by reference, in its entirety.

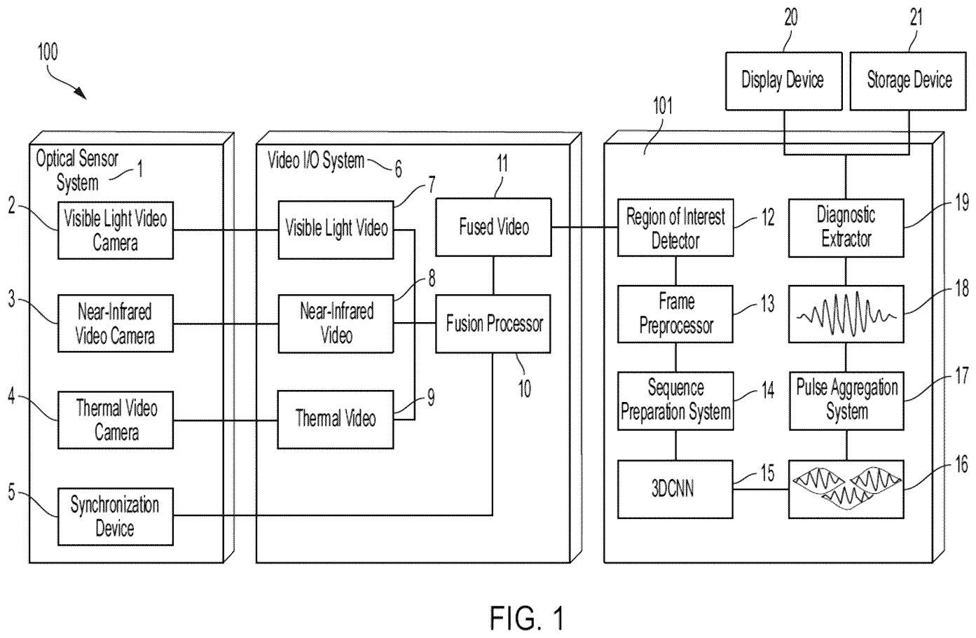

FIG. 1 illustrates a system 100 for pulse waveform estimation. System 100 includes optical sensor system 1, video I/O system 6, and video processing system 101.

Optical sensor system 1 includes one or more camera sensors, each respective camera sensor configured to capture a video stream including a sequence of frames. For example, optical sensor system 1 may include a visible-light camera 2, a near-infrared camera 3, a thermal camera 4, or any combination thereof. In the event that multiple camera sensors are utilized (e.g., single modality or multiple modality), the resulting multiple video streams may be synchronized according to synchronization device 5. Alternatively, or additionally, one or more video analysis techniques may be utilized to synchronize the video streams. Although a visible-light camera 2, a near-infrared camera 3, a thermal camera 4 are enumerated, other media devices can be used, such as a speech recorder.

Video I/O system 6 receives the captured one or more video streams. For example, video I/O system 6 is configured to receive raw visible-light video stream 7, near-infrared video stream 8, and thermal video stream 9 from optical sensor system 1. Here, the received video streams may be stored according to known digital format(s). In the event that multiple video streams are received (e.g., single modality or multiple modality), fusion processor 10 is configured to combine the received video streams. For example, fusion processor 10 may combine visible-light video stream 7, near-infrared video stream 8, and/or thermal video stream 9 into a fused video stream 11. Here, the respective streams may be synchronized according to the output (e.g., a clock signal) from synchronization device 5.

At video processing system 101, region of interest detector 12 detects (i.e., spatially locate) one or more spatial regions of interest (ROI) within each video frame. The ROI may be a face, another body part (e.g., a hand, an arm, a foot, a neck, etc.) or any combination of body parts. Example ROIs include a face, cheek, forehead, or an eye. Initially, region of interest detector 12 determines one or more coarse spatial ROIs within each video frame. Region of interest detector 12 is robust to strong facial occlusions from face masks and other head garments. Subsequently, frame preprocessor 13 crops the frame to encapsulate the one or more ROI. In some embodiments, the cropping includes each frame being downsized by bi-cubic interpolation to reduce the number of image pixels to be processed. Alternatively, or additionally, the cropped frame may be further resized to a smaller image.

Sequence preparation system 14 aggregates batches of ordered sequences or subsequences of frames from frame processer 13 to be processed. Next, 3-Dimensional Convolutional Neural Network (3DCNN) 15 receives the sequence or subsequence of frames from the sequence preparation system 14. 3DCNN 15 processes the sequence or subsequence of frames, by a 3-dimensional convolutional neural network, to determine the spatial and temporal dimensions of each frame of the sequence or subsequence of frames and to produce a pulse waveform point for each frame of the sequence of frames. 3DCNN 15 applies a series of 3-dimensional convolution, averaging, pooling, and nonlinearities to produce a 1-dimensional signal approximating the pulse waveform 16 for the input sequence or subsequences.

In some configurations, pulse aggregation system 17 combines any number of pulse waveforms 16 from the sequences or subsequences of frames into an aggregated pulse waveform 18 to represent the entire video stream. Diagnostic extractor 19 is configured to compute the heart rate and the heart rate variability from the aggregated pulse waveform 18. To identify heart rate variability, the calculated heart rate of various subsequences may be compared. Display unit 20 receives real-time or near real-time updates from diagnostic extractor 19 and displays aggregated pulse waveform 18, heart rate, and heart rate variability to an operator. Storage Unit 21 is configured to store aggregated pulse waveform 18, heart rate, and heart rate variability associated with the subject.

Additionally, or alternatively, the sequence of frames may be partitioned into a partially overlapping subsequences within the sequence preparation system 14, wherein a first subsequence of frames overlaps with a second subsequence of frames. The overlap in frames between subsequences prevents edge effects. Here, pulse aggregation system 17 may apply a Hann function to each subsequence, and the overlapping subsequences added to generate aggregated pulse waveform 18 with the same number of samples as frames in the original video stream. In some configurations, each subsequence is individually passed to the 3DCNN 15, which performs a series of operations to produce a pulse waveform for each subsequence 16. Each pulse waveform output from the 3DCNN 15 is a time series with a real value for each video frame. Since each subsequence is processed by the 3DCNN 15 individually, they are subsequently recombined.

In some embodiments, one or more filters may be applied to the region of interest. For example, one or more wavelengths of LED light may be filtered out. The LED may be shone across the entire region of interest and surrounding surfaces or portions thereof. Additionally, or alternatively, temporal signals in non-skin regions may be further processed. For example, analyzing the eyebrows or the eye's sclera may identify changes strongly correlated with motion, but not necessarily correlated with the photplethysmogram. If the same periodic signal predicted as the pulse is found on non-skin surfaces, it may indicate a non-real subject or attempted security breach.

Although illustrated as a single system, the functionality of system 100 may be implemented as a distributed system. While system 100 determines heart rate, other distributed configurations track changes to the subject's eye gaze, eye blink rate, pupil diameter, speech, face temperature, and micro-expressions, for example. Further, the functionality disclosed herein may be implemented on separate servers or devices that may be coupled together over a network, such as a security kiosk coupled to a backend server. Further, one or more components of system 100 may not be included. For example, system 100 may be a smartphone or tablet device that includes a processor, memory, and a display, but may not include one or more of the other components shown in FIG. 1. The embodiments may be implemented using a variety of processing and memory storage devices. For example, a CPU and/or GPU may be used in the processing system to decrease the runtime and calculate the pulse in near real-time. System 100 may be part of a larger system. Therefore, system 100 may include one or more additional functional modules.

Subtle quasi-periodic physiological signals such as blood volume pulse and respiration can be extracted from RGB video, enabling remote health monitoring and other applications. Advancements in remote pulse estimation-or remote photoplethysmography (rPPG)-are currently driven by supervised deep learning solutions. However, current approaches are trained and evaluated on limited benchmark datasets recorded with ground truth from contact-PPG sensors.

Remote Photoplethysmography (rPPG), or the remote monitoring of a subject's heart rate using a camera, has seen a shift from handcrafted techniques to deep learning models. While current solutions offer substantial performance gains, these models tend to learn a bias to pulse wave features inherent to the training dataset. Accordingly, the inventors develop augmentations to mitigate this learned bias by expanding both the range and variability of heart rates that the model uses while training, resulting in improved model convergence when training and cross-dataset generalization at test time. Through a 3-way cross dataset analysis, a reduction in mean absolute error from over 13 beats per minute to below 3 beats per minute is demonstrated.

Measuring a subject's heart rate is an important component of physiological monitoring. While methods such as photoplethysmography (PPG) exist for contact heart rate monitoring, a push has been made for non-contact remote photo-plethysmography (rPPG). rPPG is cheaper, requiring a commodity camera rather than a specialized pulse oximeter, and it is contact-free, allowing for applications in new contexts.

Initial techniques for rPPG employed algorithms involving a multi-stage pipeline. While these techniques may be highly accurate, their performance is adversely affected by dynamics common in videos such as motion and illumination changes. More recently, deep learning methods have been applied to rPPG, many of them outperforming handcrafted techniques. While deep learning techniques have benefits, they suffer drawbacks as well in terms of generalization. It has been shown that the learned priors in deep learning rPPG models are strong enough to predict a periodic signal in situations where a periodic signal is not present in the input-a relevant attack scenario. It is demonstrated that a deep learning rPPG model may be biased toward predicting heart rate features such as the frequency bands and rates of change that appear in its training data, and therefore struggle to generalize to new situations.

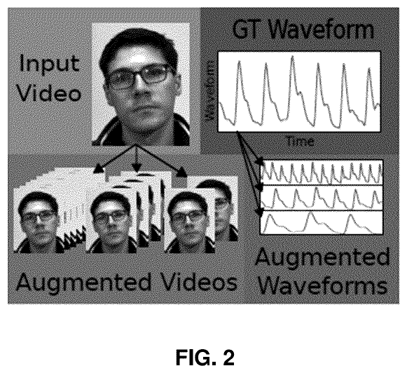

Training of rPPG models incorporates various types of data augmentations in the spatial domain. In the embodiments, the data in the temporal domain is augmented-injecting synthetic data representing a wide spectrum of heart rates, thus enabling models to better respond to unknown heart rates. The embodiments are evaluated in a cross-dataset setup comprising significant differences between heart rates in the training and test subsets. For example, FIG. 2 illustrates an overview of the augmentations targeting the temporal domain according to an example embodiment.

There has been broad interest in rPPG, with applications including detection of heart arrhythmias such as atrial fibrillation, deepfake detection, and affective computing. However, early techniques were not robust to motion. The emergence of practical deep learning methods has enabled new methods for rPPG estimation, such as DeepPhys, a convolutional neural network (CNN) model which effectively predicts pulse waveform derivatives based on adjacent video frames. Additionally, a 3DCNN based approach has been developed for predicting the pulse waveform from video data.

Cross-dataset generalization is a common concern with deep learning techniques, specifically in that deep learning rPPG techniques tend to perform suboptimally when working outside of the heart rate range of the training set. In the various embodiments, speed and modulation augmentations for 3DCNN based models are provided, showing that this consideration mitigates much of the cross dataset performance loss experienced by current models.

A variety of computer systems, devices, methods, and non-transitory computer-readable instructions may be utilized for rPPG analysis. The example embodiments utilize the RPNet architecture, which is a 3DCNN-based approach. For example, the network architecture may be composed of 3D convolutions with max and global pooling layers for dimension reduction. The network may consume 64×64 pixel video over a 136-frame window, outputting an rPPG signal of 136 samples.

The preprocessing pipeline consists of the following steps. First, facial landmarks are obtained at each frame in the dataset using the MediaPipe Face Mesh tool. Second, the face is cropped at the extreme points of the landmarks, padded by 30% on the top and 5% on the sides and bottom, and the shortest dimension is extended to make the crop square. Third, the cropped portion is scaled to 64×64 pixels using cubic interpolation.

When a cross-dataset analysis is performed, the frame rate of all videos is reduced to the lowest common denominator, e.g., 30 FPS. This only affects the DDPM dataset, which is recorded at 90 FPS. The conversion is executed before the cropping step by taking the average pixel value over sets of three frames. The “averaging” technique is used rather than skipping frames in order to better emulate a slower camera shutter speed.

RPNet outputs rPPG waves in 136-frame chunks with a stride of 68 frames. These parameters can be used so that the model is small enough to fit on the used GPUs. To reduce edge effects, a Hann window may be applied to the overlapping segments and add them together, thus producing a single waveform.

To enable the determination of heart rates, a Short-Time Fourier Transform (STFT) of the output waveform is used with a window size of 10 seconds and a stride of 1 frame, thus enabling the use of the embodiments in application scenarios tolerant of a 10-second latency. The waveform may be padded with zeros such that the bin width in the frequency domain is 0.001 Hz (0.06 beats per minute (BPM)) to reduce quantization effects. Additionally, the highest peak is selected in the range of 0.66 and 3 Hz (i.e., 40 and 180 BPM) as the inferred heart rate.

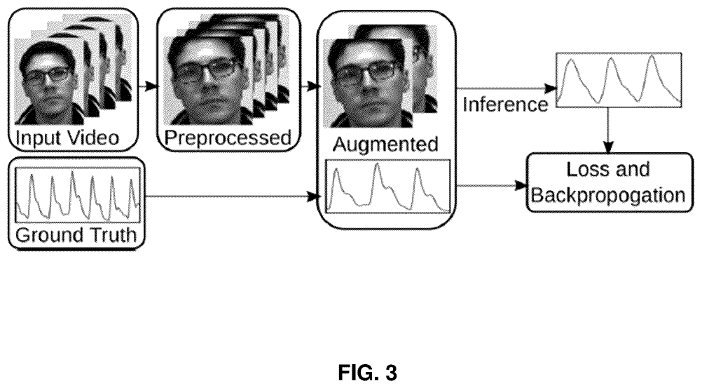

The temporal aspect of the training data is augmented, affecting alternatively the heart rate or speed, and the change in heart rate or modulation. FIG. 3 illustrates an overview of the temporal augmentation framework according to an example embodiment. FIG. 3 shows how it fits into the training protocol.

To apply the speed augmentation, first randomly select a target heart rate between 40 and 180 BPM (i.e., the desired range of heart rates for which the model will be sensitive). This is set to be the same range as the peak selection used in the postprocessing step so that the model will be trained to predict the same heart rates that the rest of the system is designed to handle.

Second, the ground truth heart rate (i.e., obtained using the same STFT technique outlined above) is averaged over the 136 frame clip, as the source heart rate. Next, the length of data centered on the source clip is calculated to be:

└136×HRtarget/HRsource].

Third, the data in the source interval is interpolated such that it becomes 136 frames long. This process is applied to both the video clip and the ground truth waveform.

To apply the modulation augmentation, randomly select a modulation factor f based on the ground truth heart rate such that when the clip speeds up or slows down by a factor of f, the change in heart rate is no more than 7 BPM per second. This parameter was selected based on the maximum observed change in heart rate in the DDPM dataset. Further, constrain the modulation such that the video clip is modulated linearly by the selected factor over its duration, i.e. for normalized heart rates s and e at the start and end of the clip respectively, the normalized heart rate at each frame x in the n frame clip (set to 136 as in Section 3.1) is:

$$nHR ( x ) = s + \frac{x ( e - s )}{n} \qquad ( 1 )$$

where s=2/(1+f) and e=sf. Then, integrate nHR to generate a function yielding the positions P(x) along the original clip at which to interpolate:

$$P ( x ) = xs + \frac{{x}^{2} ( e - s )}{2 n} + c \qquad ( 2 )$$

where c=0 due to indexing starting at 0. Finally, linearly interpolate the n frames from the original video clip at every position P(x) for all x in the range [0 . . . n], thus yielding the modulated video clip. Optionally, horizontal flip, illumination, and Gaussian noise spatial augmentations may be used.

A variety of metrics may be used for evaluation. These metrics utilize either the pulse waveform (provided as ground truth or inferred by RPNet) or the heart rate. If the lengths of the ground truth and predicted waves differ (as is the case if the ground truth wave is not a multiple of 68 frames, i.e. the stride used for RPNet), then data points from the end of the ground truth wave are removed such that they have the same length.

Each evaluation metric is calculated over each video in the dataset independently, the results of which are averaged. Example evaluation metrics will now be described.

The Mean Error (ME) captures the bias of the method in BPM, and is defined as follows:

$$ME = \frac{1}{N} {\sum }_{i = 1}^{N} ( {HR}_{i}^{′} - {HR}_{i} ) \qquad ( 3 )$$

where HR and HR′ are the ground truth and predicted heart rates, respectively, where each contained index is the heart rate obtained from the STFT window, and N is the number of STFT windows present. Many rPPG methods omit an analysis based on ME since it is often close to zero due to positive and negative errors canceling each other out. However, ME is valuable for gauging the bias of a model in a cross-dataset analysis by explaining how the model is failing, i.e. whether the predictions are simply noisy or if they are shifted relative to the ground truth.

The Mean Absolute Error (MAE) captures an aspect of the precision of the method in BPM, and is defined as follows:

$$MAE = \frac{1}{N} {\sum }_{i = 1}^{N} \vert {HR}_{i}^{′} - {HR}_{i} \vert \qquad ( 4 )$$

The Root Mean Squared Error (RMSE) is similar to MAE, but penalizes outlier heart rates more strongly:

$$RMSE = \sqrt{\frac{1}{N} {\sum }_{i = 1}^{N} {( {HR}_{i}^{′} - {HR}_{i} )}^{2}} \qquad ( 5 )$$

The waveform correlation, rwave, is the Pearson's r correlation coefficient between the ground truth and predicted waves. When performing an inter-dataset analysis, the rwave value is further maximized by varying the correlation lag between ground truth and predicted waves by up to 1 second (30 data points) in order to compensate for differing synchronization techniques between datasets.

For cross dataset analysis, three rPPG datasets were used as an example, chosen to contain a wide range of heart rates: PURE, UBFC-rPPG, and DDPM. Key statistics for these three datasets are summarized in Table 1.

TABLE 1
Average duration, heart rate (HR) in BPM calculated using
the STFT, and average within-session standard deviation
in HR within a 60 second window and a stride of 1 frame,
for PURE, UBFC-rPPG, and DDPM. The 95% confidence intervals
are calculated across sessions in the dataset.

| Dataset | Duration (s) | HR Avg | HR SD |
|---|---|---|---|
| PURE | 68.307 ± 1.502 | 69.200 ± 6.026 | 1.638 ± 0.2682 |
| UBFC | 64.964 ± 1.516 | 100.801 ± 5.056 | 3.016 ± 0.525 |
| DDPM | 656.464 ± 22.310 | 96.982 ± 4.186 | 4.000 ± 0.286 |

The PURE dataset is useful for cross-dataset analysis for two key reasons. First, it has the lowest average heart rate of the three datasets, being about 30 BPM lower than the other two. Second, it has the lowest within-subject heart rate standard deviation.

The UBFC-rPPG dataset (shortened to UBFC) features subjects playing a time-sensitive mathematical game which caused a heightened physiological response. UBFC has the highest average heart rate of the three datasets and more heart rate variability than PURE, but less variability than DDPM.

The DDPM dataset is the largest of the compared datasets, with recorded sessions lasting nearly 11 minutes on average. It also features the most heart rate variability of the three, with a heart rate standard deviation of about 4 BPM. This is due to stress-inducing aspects (mock interrogation with forced deceptive answers) in the collection protocol of DDPM. Due to noise in the ground truth oximeter waveforms, 10 second segments in DDPM where the heart rate changes by more than 7 BPM per second may be masked out.

Training. For each of the three datasets, randomly partition the videos into five subject-disjoint sets, three of which are merged to generate splits for training, validation, and testing at 3/1/1 ratios, for example. Then, rotate the splits to generate five folds for cross-validation. For example, train for 40 epochs using the negative Pearson loss function and the Adam optimizer configured with a 0.0001 learning rate. Models are selected based on minimum validation loss.

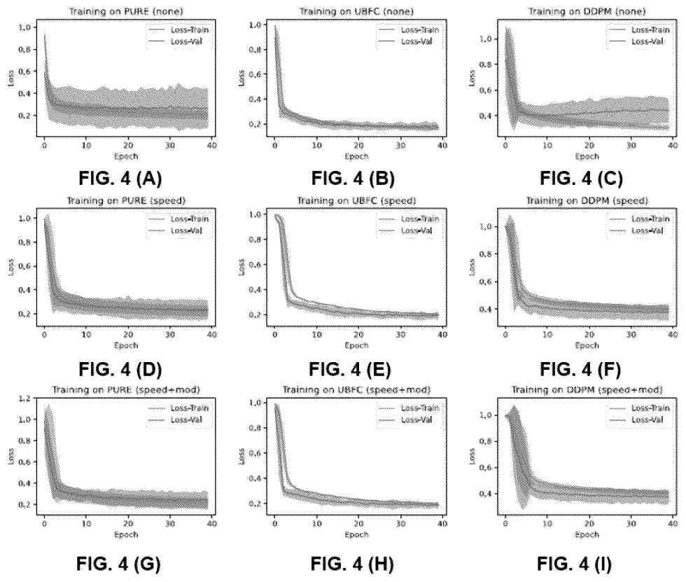

FIGS. 4(A)-(I) illustrate training and validation losses when training RPNet on the three datasets and applying three augmentation settings: none, speed, and speed+mod. Utilizing any sort of temporal augmentation causes the validation loss to converge with tighter confidence intervals. This is especially evident when training on the PURE dataset where the median validation loss confidence interval without temporal augmentations (FIG. 4A) drops from ±0.174 to ±0.081 and ±0.078 with speed and speed+mod augmentations, respectively (FIGS. 4D and 4G). Furthermore, while it is apparent from FIG. 4C that training over DDPM without temporal augmentations may lead to overfitting, both temporal augmentation settings appear to avoid this problem (FIGS. 4F and 4I).

Across all combinations of augmentations and datasets, the validation loss converges to a lower value when temporal augmentations are used than when they are not. This is likely because the models are forced to generalize when the range and variability of heart rates they are exposed to is increased, limiting the effectiveness of simply memorizing a signal which looks like a heart rate and replaying it at a frequency common to the dataset.

The various embodiments trained and tested RPNet on each of the three datasets, both in a within dataset analysis (3 training-testing configurations with PURE-PURE, UBFC-UBFC, and DDPM-DDPM), and with a cross-dataset analysis (6 training-testing configurations with PURE-UBFC, PURE-DDPM, UBFC-PURE, UBFC-DDPM, DDPM-PURE, and DDPM-UBFC). Furthermore, three temporal augmentation settings were used, namely no temporal augmentation (none), speed augmentation (speed), and speed plus modulation augmentation (speed+mod). The results for the within-dataset analysis are shown in Table 2 and for the cross-dataset analysis are shown in Table 3.

TABLE 2
Results for the 9 within-dataset combinations of dataset and the temporal
augmentations used. Heart rate metrics (ME, MAE, and RMSE) have units
of BPM, and rwave is Pearson's r correlation over pulse waveforms.

| Dataset | Augmentations | ME | MAE | RMSE | r_{wave} |
|---|---|---|---|---|---|
| PURE | none | −0.516 ± 1.814 | 1.176 ± 1.891 | 1.872 ± 3.067 | 0.694 ± 0.253 |
| PURE | speed | −0.012 ± 0.461 | 0.694 ± 0.566 | 1.222 ± 1.456 | 0.753 ± 0.087 |
| PURE | speed + mod | 0.006 ± 0.389 | 0.639 ± 0.482 | 1.130 ± 1.347 | 0.752 ± 0.089 |
| UBFC | none | 0.922 ± 2.215 | 1.432 ± 2.201 | 2.238 ± 2.630 | 0.803 ± 0.024 |
| UBFC | speed | 0.016 ± 0.384 | 0.616 ± 0.201 | 1.346 ± 0.746 | 0.793 ± 0.020 |
| UBFC | speed + mod | 0.091 ± 0.139 | 0.502 ± 0.121 | 0.993 ± 0.335 | 0.798 ± 0.024 |
| DDPM | none | −1.443 ± 5.725 | 4.167 ± 4.680 | 6.907 ± 6.504 | 0.569 ± 0.070 |
| DDPM | speed | −0.773 ± 2.036 | 3.230 ± 2.267 | 5.897 ± 4.671 | 0.584 ± 0.052 |
| DDPM | speed + mod | −1.048 ± 1.434 | 2.981 ± 1.738 | 5.485 ± 3.412 | 0.587 ± 0.057 |

While the temporal augmentations were intended to improve cross-dataset performance, a slight performance boost occurred in the within-dataset case. As shown in Table 2, all metrics except rwave on UBFC exhibited better performance when temporal augmentations were employed. However, in these cases the performance boost is slight, often falling within the 95% confidence intervals of the results without augmentation.

Turning to the cross-dataset case shown in Table 3, it was found that training on a dataset with higher heart rate variability and testing on a dataset with lower heart rate variability tends to produce better results than the reverse. This is especially evident in cross dataset cases involving DDPM, which has the highest heart rate variability as measured by heart rate standard deviation in Table 1.

TABLE 3
Results for the 18 cross-dataset combinations of train dataset, test dataset,
and temporal augmentations used. Heart rate metrics (ME, MAE, and RMSE) have
units of BPM, while rwave is Pearson's r correlation over pulse waveforms.

| Train | Test | Augmentations | ME | MAE | RMSE | r_{wave} |
|---|---|---|---|---|---|---|
| PURE | UBFC | none | −13.082 ± 12.972 | 13.690 ± 12.847 | 19.320 ± 13.359 | 0.532 ± 0.136 |
| PURE | UBFC | speed | −3.340 ± 2.998 | 4.703 ± 3.083 | 9.219 ± 4.645 | 0.590 ± 0.102 |
| PURE | UBFC | speed + mod | −1.491 ± 0.583 | 2.251 ± 0.671 | 5.191 ± 1.559 | 0.636 ± 0.053 |
| PURE | DDPM | none | −27.633 ± 8.058 | 32.360 ± 3.934 | 38.397 ± 3.052 | 0.182 ± 0.015 |
| PURE | DDPM | speed | −10.926 ± 11.184 | 24.343 ± 4.140 | 33.410 ± 3.694 | 0.221 ± 0.032 |
| PURE | DDPM | speed + mod | 6.436 ± 4.870 | 33.620 ± 2.018 | 42.494 ± 2.829 | 0.150 ± 0.015 |
| UBFC | PURE | none | 9.657 ± 3.971 | 11.532 ± 2.710 | 14.791 ± 2.751 | 0.619 ± 0.021 |
| UBFC | PURE | speed | 0.864 ± 1.074 | 2.196 ± 0.921 | 3.758 ± 1.289 | 0.671 ± 0.043 |
| UBFC | PURE | speed + mod | 0.938 ± 0.720 | 2.535 ± 0.920 | 4.246 ± 1.275 | 0.625 ± 0.025 |
| UBFC | DDPM | none | −5.569 ± 4.479 | 14.947 ± 2.231 | 20.738 ± 2.366 | 0.264 ± 0.028 |
| UBFC | DDPM | speed | −4.240 ± 6.961 | 18.574 ± 2.707 | 28.082 ± 3.056 | 0.251 ± 0.020 |
| UBFC | DDPM | speed + mod | 11.258 ± 4.904 | 32.914 ± 0.769 | 41.698 ± 0.834 | 0.174 ± 0.010 |
| DDPM | PURE | none | 26.092 ± 14.065 | 26.660 ± 13.435 | 30.915 ± 13.164 | 0.437 ± 0.099 |
| DDPM | PURE | speed | 1.256 ± 1.563 | 2.208 ± 1.824 | 3.905 ± 2.996 | 0.686 ± 0.061 |
| DDPM | PURE | speed + mod | 1.338 ± 1.477 | 2.509 ± 1.776 | 4.441 ± 2.991 | 0.673 ± 0.058 |
| DDPM | UBFC | none | −0.358 ± 0.863 | 1.963 ± 1.135 | 3.745 ± 1.931 | 0.699 ± 0.050 |
| DDPM | UBFC | speed | −0.431 ± 0.177 | 1.311 ± 0.282 | 3.140 ± 0.654 | 0.711 ± 0.028 |
| DDPM | UBFC | speed + mod | −0.563 ± 0.383 | 1.160 ± 0.393 | 2.906 ± 1.112 | 0.734 ± 0.029 |

As shown in the ME column of Table 3, it was observed that when training and testing between datasets of different heart rates without temporal augmentations, the bias as reflected by ME is strong, with UBFC-PURE yielding the ME closest to zero at over 9 BPM. Furthermore, these models are biased in the direction of the training dataset's mean heart rate, i.e. training on PURE which has relatively low heart rates results in a negative ME on UBFC and DDPM, while training on UBFC or DDPM results in a positive ME when testing on PURE. However, applying the speed augmentation causes ME to be much closer to zero than when no such augmentation is used. This is because the speed augmentation is intended to mitigate the heart rate bias inherent in the training dataset, thus causing it to generalize to any heart rates seen in the augmented training regime rather than simply those present in the dataset. With the mitigation of heart rate bias as reflected by improved ME scores, improvement in MAE and RMSE occurs in most cases. Furthermore, a boost in rwave was observed indicating that the models more faithfully reproduce the waveforms with low noise.

The modulation augmentation is intended to boost performance when training on a dataset with low heart rate variability such as PURE and testing on a dataset with high variability such as UBFC and DDPM. Modulation boosts performance for PURE-UBFC, though even with modulation PURE-DDPM fails to generalize. With the possible exception of DDPM-UBFC, the modulation augmentation does not positively impact cases when the training dataset already contains high heart rate variability, as is the case with UBFC and DDPM.

Poor results occurred in both cross dataset experiments where DDPM is the test dataset. Of those, the same trend was observed in PURE-DDPM as in other cases, i.e. that models trained with speed augmentations outperform those without, albeit in this case the performance is still quite poor. In UBFC-DDPM, models trained without speed augmentations achieve better results than with speed augmentations, which is a break from the trend observed in all other cases. Furthermore, whereas in other cases high MAE and RMSE errors are largely explained by bias as reflected in ME, this case has a relatively low ME relative to MAE and RMSE. In this case since the average heart rate between UBFC and DDPM is relatively close (differing by less than 4 BPM), overfitting to this band of heart rates is actually beneficial for the cross dataset analysis.

Furthermore, the “zero-effort” error rates achieved by a model which simply predicts the average heart rate for the dataset (97 BPM as in Table 1) has comparable error rates to UBFC-DDPM (MAE and RMSE are 17.804 and 22.113 respectively). These zero-effort results for the three datasets are reported in Table 4.

TABLE 4
Zero-effort errors obtained by predicting the average heart
rate of the dataset for all subjects. In all cases ME is 0.

| Dataset | MAE | RMSE |
|---|---|---|
| PURE | 15.847 | 23.054 |
| UBFC | 14.085 | 17.256 |
| DDPM | 17.804 | 22.113 |

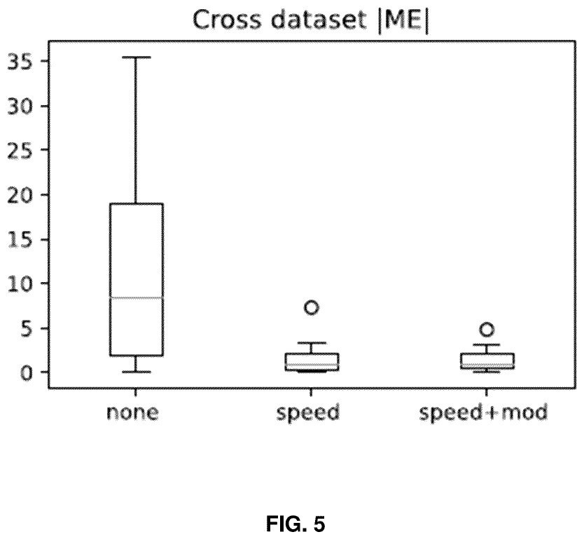

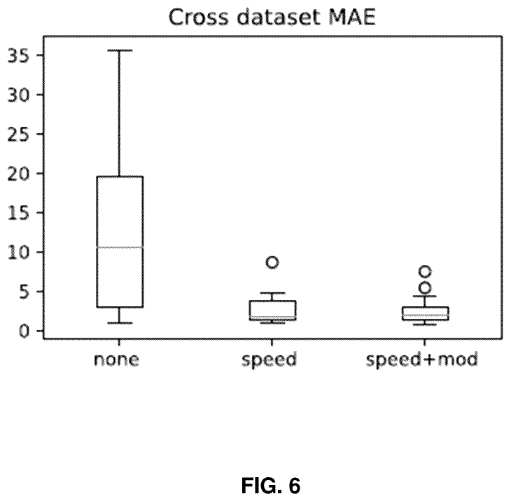

The cross dataset results are summarized in Table 5. The 95% confidence interval is calculated across 4 cross dataset combinations (omitting the cases when testing on DDPM as no models generalized) and 5 training folds. Combining both speed and modulation losses yields optimal performance on all metrics. The box plots in FIG. 5 and FIG. 6 further demonstrate the reason why the temporal augmentations outperform the case without augmentations. FIG. 5 illustrates that speed augmentations reduce learned bias as reflected by a reduced ME in cross dataset analysis between datasets with differing heart rate bands according to an example embodiment. FIG. 6 illustrates that speed augmentations improve the accuracy of the model, reflected by an improved MAE. Accordingly, the bias of the model to predict heart rates similar to its training dataset has been significantly reduced, as is most clearly seen in the reduced absolute ME shown in FIG. 5, and the improved MAE shown in FIG. 6.

TABLE 5
Summaries of cross dataset performance under speed augmentation settings,
omitting PURE-DDPM and UBFC-DDPM where no models succeed in generalizing.
The absolute value of ME metrics before averaging is used.

| Augmentation | \|ME\| | MAE | RMSE | r_{wave} |
|---|---|---|---|---|
| none | 12.349 ± 5.546 | 13.460 ± 5.335 | 17.192 ± 5.720 | 0.572 ± 0.056 |
| speed | 1.502 ± 0.803 | 2.604 ± 0.884 | 5.005 ± 1.536 | 0.664 ± 0.031 |
| speed + mod | 1.373 ± 0.570 | 2.501 ± 0.784 | 4.830 ± 1.174 | 0.677 ± 0.025 |

The augmentations described herein are generally applicable to deep learning based rPPG as a whole, as these augmentation techniques may be implemented as a training framework for any model architecture that trains based on video inputs (or other data inputs) to produce waveform outputs.

The importance of temporal speedbased augmentations for the cross-dataset generalization of deep learning rPPG methods is demonstrated. In addition, a system for training deep learning rPPG models using two variants of this augmentation method is provided, i.e. speed augmentation affecting the heart rate, and modulation affecting the change in heart rate. These augmentations may be applied to any deep learning rPPG system which produces a pulse waveform from video inputs.

It will be apparent to those skilled in the art that various modifications and variations can be made in the generalization in cross-dataset remote photoplethysmography of the present invention without departing from the spirit or scope of the invention. Thus, it is intended that the present invention cover the modifications and variations of this invention provided they come within the scope of the appended claims and their equivalents.

What is claimed is:

1. A computer-implemented method for determining a physiological signal from a video stream, the computer-implemented method comprising:
capturing the video stream of a subject, the video stream including a sequence of frames;
processing each frame of the video stream to identify a facial portion of the subject in each frame;
determining a periodic physiological signal of the subject from the video stream using a plurality of datasets in which one or more augmentations were applied to the plurality of datasets.

2. The computer-implemented method according to claim 1, wherein the plurality of datasets includes ground truth data and previously captured video data.

3. The computer-implemented method according to claim 2, wherein the ground truth data is captured using pulse oximeter.

4. The computer-implemented method according to claim 2, wherein the one or more augmentations is applied to the ground truth data and the previously captured video data.

5. The computer-implemented method according to claim 1, wherein the one or more augmentation include at least one of horizontal flip, illumination, and Gaussian noise.

6. The computer-implemented method according to claim 1, wherein the periodic physiological signal is heart rate.

7. The computer-implemented method according to claim 1, wherein the facial portion is cropped to 64×64 pixels.

8. The computer-implemented method according to claim 1, wherein the video stream includes one or more of a visible-light video stream, a near-infrared video stream, a longwave-infrared video stream, a thermal video stream, and an audio stream of the subject.

9. The computer-implemented method according to claim 1, further comprising cropping each frame of the media stream to encapsulate a region of interest that includes one or more of a face, cheek, forehead, or an eye.

10. The computer-implemented method according to claim 1, further comprising instructions for:
combining at least two of a visible-light video stream, a near-infrared video stream, and a thermal video stream into a fused video stream.

11. A system for determining a physiological signal from a video stream, the system comprising:
a processor; and
a memory storing one or more programs for execution by the processor, the one or more programs including instructions for:
capturing the video stream of a subject, the video stream including a sequence of frames;
processing each frame of the video stream to identify a facial portion of the subject in each frame;
determining a periodic physiological signal of the subject from the video stream using a plurality of datasets in which one or more augmentations were applied to the plurality of datasets.

12. The system according to claim 11, wherein the plurality of datasets includes ground truth data and previously captured video data.

13. The system according to claim 12, wherein the ground truth data is captured using pulse oximeter.

14. The system according to claim 12, wherein the one or more augmentations is applied to the ground truth data and the previously captured video data.

15. The system according to claim 11, wherein the one or more augmentation include at least one of horizontal flip, illumination, and Gaussian noise.

16. The system according to claim 11, wherein the periodic physiological signal is heart rate.

17. The system according to claim 11, wherein the facial portion is cropped to 64×64 pixels.

18. The system according to claim 11, wherein the video stream includes one or more of a visible-light video stream, a near-infrared video stream, a longwave-infrared video stream, a thermal video stream, and an audio stream of the subject.

19. The system according to claim 11, further comprising cropping each frame of the media stream to encapsulate a region of interest that includes one or more of a face, cheek, forehead, or an eye.

20. The system according to claim 11, further comprising instructions for:
combining at least two of a visible-light video stream, a near-infrared video stream, and a thermal video stream into a fused video stream.
